(19) United States
(12) Patent Application Publication
(10) Pub. No.: US 2009/0143844 A1
(43) Pub. Date: Jun. 4, 2009

(54) HOSE MANAGEMENT FOR CONVECTIVE DEVICES

(75) Inventor: Karl H. Cazzini, Orchard Park, NY (US)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(73) Assignee: Gaymar Industries, Inc., Orchard Park, NY (US)

(21) Appl. No.: 11/946,929

(22) Filed: Nov. 29, 2007

(51) Int. Cl. A61F 7/08

(52) U.S. Cl. 607/104

(57) ABSTRACT

The present invention is directed to a convective thermal unit. The convective thermal unit has the conventional blower that directs ambient air to a heating element, the heating element heats the air and the heated air is directed into a conduit. The conduit directs the heated air into a receiving unit like a blanket positioned over a patient. A difference between the prior art and the present invention is the incorporation of a shape memory polymer and/or alloy material into the conduit to ensure the conduit does not contact the ground when the convective thermal unit is not being used or not providing the desired thermal energy.

### FIELD OF THE INVENTION

The present invention is directed to controlling a position of a hose.

### BACKGROUND OF THE INVENTION

In U.S. Pat. No. 5,747,993, Jacobson et al. wrote about controlling a hose's position. In particular, Jacobson et al. wrote, “Disposed on one side of [a] bar . . . is a strip . . . of shape memory alloy which has the capability of changing its shape upon the application of external heat or electric current (which generates internal heat) to some other shape and then assuming the original shape when cooled or electric current is removed and the heat dissipates. Example of such shape memory alloy is nitonol comprised of about 50 percent nickel and 50 percent titanium. The bar . . . is made of a laterally flexible material such as ceramic, metal or plastic, so that when the shape memory alloy strip . . . is caused to change shape, such as contract along its length, the bar will be caused to bend . . . .

In this embodiment, two flexible tubes . . . are anchored respectively on bases . . . . The free ends of the tubes are positioned to mate together in a colinear fashion to seal the inside of the tubes from the outside when the tubes are undeflected. An access port . . . is formed in the tube . . . to allow introduction of fluid to the inside of the tubes. Of course, such access could be provided through the other tube . . . or through the bases . . . . Strips of shape memory alloy are disposed on the upper sides of the tubes . . . and are selectively heated by a current source to cause the tubes to deflect or bend upwardly . . . . When such deflection occurs, the ends of the tubes . . . are exposed to allow escape of fluid which has been introduced into the insides of the tubes . . . . When current to the strips of shape memory alloy is terminated so that the strips cool, the strips return to their original shape causing the tubes to deflect back to their original colinear position to again seal the inside of the tubes from the outside and prevent further outflow of fluid.”

As described above, shape memory alloys have been used in association with hoses. The shape memory alloys have not, however, been used to control (a) the position of a hose to prevent contact with the ground and/or (b) fluid turbulence in the hoses.

### SUMMARY OF THE INVENTION

The present invention is directed to a convective thermal unit. The convective thermal unit has a conventional blower and a heating element. The conventional blower directs ambient air to the heating element. The heating element heats the air and the heated air is directed into a conduit. The conduit directs the heated air into a receiving unit, for example, a blanket positioned over a patient. A difference between the prior art and the present invention is the incorporation of a shape memory polymer and/or alloy material into the conduit to ensure the conduit does not contact the ground when the convective thermal unit is not being used and/or not providing a minimum desired thermal energy.

### BRIEF DESCRIPTION OF THE FIGURES

The figures and the descriptions set forth in this document are examples of the present invention and not limit the breadth and scope of the present invention.

FIG. 1 illustrates a fluid blanket warming system having a convective thermal unit and a blanket unit.

FIG. 2 illustrates a cross-sectional view of the convective thermal unit.

FIG. 3 illustrates a cross-sectional view of a portion of a conduit of the convective thermal unit interconnected to the blanket unit.

FIG. 4 illustrates a view of FIG. 3 taken along the lines 4-4.

FIG. 5 illustrates an electrical schematic of one embodiment of the convective thermal unit.

FIG. 6 illustrates a portion of the conduit having the shape memory polymer and/or alloy positioned on the exterior surface of the conduit.

FIG. 7 illustrates a portion of the conduit having the shape memory polymer and/or alloy positioned in the interior surface of the conduit.

FIG. 8 illustrates a portion of the conduit having the shape memory polymer and/or alloy embedded in the material of the conduit.

FIG. 9 illustrates the present invention with the conduit in a compressed position.

### DETAILED DESCRIPTION OF THE INVENTION

The present invention can be directed to a fluid blanket warming system 2 having a convective thermal unit 100 and a blanket unit 4. An example of a convective thermal unit 100 is sold by Gaymar Industries, Inc. under the THERMACARE trademark. Other examples of convective thermal units 100 include and are not limited to Azirant's Model 505 Temperature Management Unit and Model 750 Temperature Management Unit. Each one of these convective thermal units 100 takes ambient air and heats the ambient air to a desired temperature. The heated air at a desired temperature is directed into a hose 8. From the hose 8, the heated air is directed into the convective blanket 4 that disperses the heated air toward a patient.

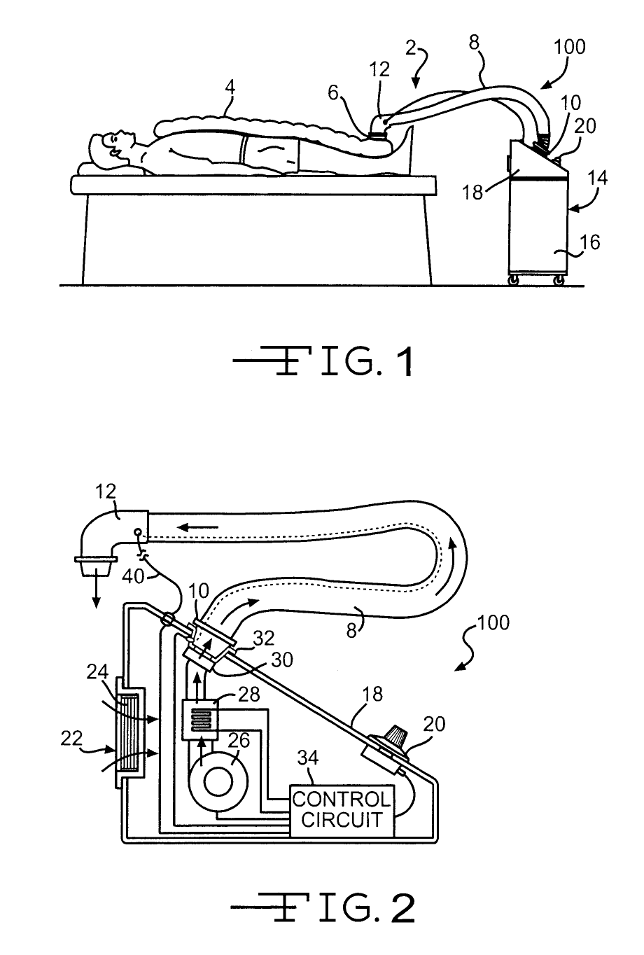

An example of the blanket unit 4 is sold by Gaymar Industries, Inc. Examples of such thermal blankets are disclosed in Augustine Medical, Inc. v. Gaymar Indus., Inc., 181 F.3d 1291, 50 USPQ2d 1900 (Fed. Cir. 1999). In that decision, the Federal Circuit wrote, “[Gaymar's convective] blankets feature an inflatable quilt-like structure. The [Gaymar] blankets attach two sheets of the same amount of flexible, lightweight material around their periphery and at various spots along their surfaces. In operation, heated air flows onto a patient's body from holes in the undersurface of [Gaymar's] blankets, but the blankets do not form a self-supporting or Quonset hut-like structure. Instead, [Gaymar's] blankets lie flat when inflated on a flat surface and rest substantially on a patient when in use. Gaymar began selling forced-air blankets in March 1992.” The blanket unit 4 is sometimes referred to as a thermal blanket, inflatable blanket or air blanket and can be subjectively configured to address substantially all or selected portions of a patient's body. The blanket unit 4 can be configured with seams and can have air slits or holes on the underside to deliver the fluid such as heated air or other gases to the patient when inflated. Generally, the blanket unit 4 provides an air plenum of approximately a hollow core for receiving the heated air and distributing it to the patient's body. Sometimes, the air blanket is divided, however, into segments or conduits to assist in erecting the air blanket as a canopy across the patient's body. The specific form of the blanket as it is adapted to a portion of the patient's body can be a feature of the present invention and numerous different examples exist and are well known by persons of skill in this field. Alternative embodiments can have the blanket positioned below the patient and/or to a patient's side. Thus, the blanket shown in FIG. 1 is for schematic purposes only and does not represent any limitation to the air blankets or thermal blankets that can be utilized in the present invention.

The flexible conduit 8 is usually formed of a flexible plastic material that can be corrugated and/or straight, and has a first coupler 10 at one end of the conduit configuration, and a second coupler 12 at the other end of the conduit configuration. A heat source 14 includes a heater housing or cabinet 16 that can be mounted for portability with wheels at the bottom. The upper portion of the housing 16 supports a console 18 with operator temperature controls 20.

Referring to FIG. 2, the console 18 includes an inlet port 22 with an optional filter 24 that allows ambient air to be drawn into the cavity of the console 18. A blower unit 26 creates a positive pressure to direct the ambient air to a heater unit 28. In the embodiment of FIG. 2, ambient air is being used as the heated fluid for application to the thermal blanket 4. It is possible, however, to provide other gases, if desired. Various configurations of blower scroll compressor, fans, etc. can be used to provide a positive air pressure. Likewise, the heater unit can also have different configurations than the resistance heater coils shown.

Downstream of the heater unit 28, a second gas filter 30 can be positioned next to a coupler 32 on the console 18. The coupler 32 connects the conduit 8 to the console 18. The second filter 30 provides extra filtration but could be eliminated, if desired. The coupler 32 is mounted on the console 18, and the first coupler 10 on the flexible conduit 8 can be removably connected to coupler 32.

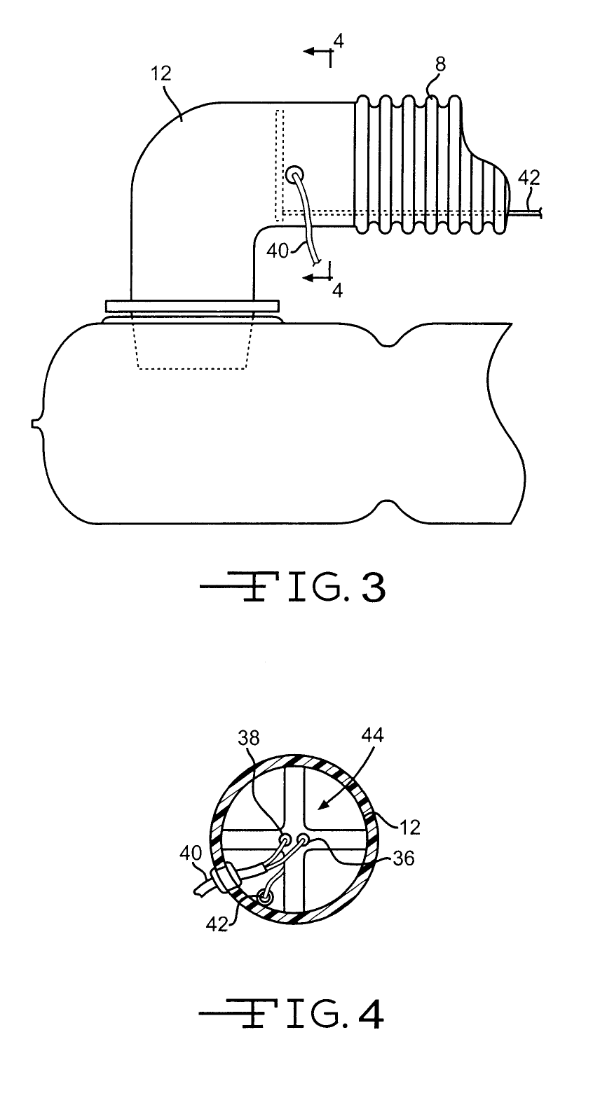

A control circuit 34 is connected, respectively, to the blower unit 26, the heater unit 28, and a first temperature sensor unit 36. In a first embodiment, the first temperature sensor unit 36 can be mounted within a housing in the form of the second coupler 12, as seen in FIGS. 3 and 4, near the heater 28, in the blanket 4, in the hose 8, or near the first coupler 10.

A second temperature sensor unit 38 can also be mounted on the housing of the second coupler 12, near the first coupler 10, in the blanket 4, in the hose 8, or near the heater 28 and connected to the control circuit 34 to provide a backup or redundancy for safety purposes, as will be subsequently described.

As shown in FIG. 2, the second sensor unit 38 can have an exterior electrical connector line 40 that can be mounted by plug into a receptacle on the exterior of the console housing 18. Also, as shown in FIGS. 3 and 4, the first sensor unit 36 is connected to an electrical connector line 42 that can travel along an interior of the flexible conduit 8. As can be appreciated, the electrical connector line 40 can also be mounted to extend along the interior of the flexible conduit 8 and, if desired, they can be fastened or adhered to the internal surface of the conduit 8. As shown in FIG. 2, the connector line 42 from the first sensor unit 36 can connect with an appropriate plug or receptacle in the coupler 32 on the console 18 for connection with the control circuit 34.

By providing the first sensor 36 and the second sensor 38 in the second coupler 12, the temperature of the heated air, as it is delivered to the thermal blanket 4, can be measured. Any bends in the flexible conduit 8 that may effect a temperature drop, may occur upstream of the second coupler 12 and heat loss from the flexible conduit 8 will be accounted for.

The first sensor unit 36 and the sensor unit 38 may be any electrical or electronic device for temperature sensing, such as a thermal couple, thermistor, resistive temperature device (RTD), semiconductor diodejunction, or integrated circuit temperature sensor with and without integrated controller or signal conditioner.

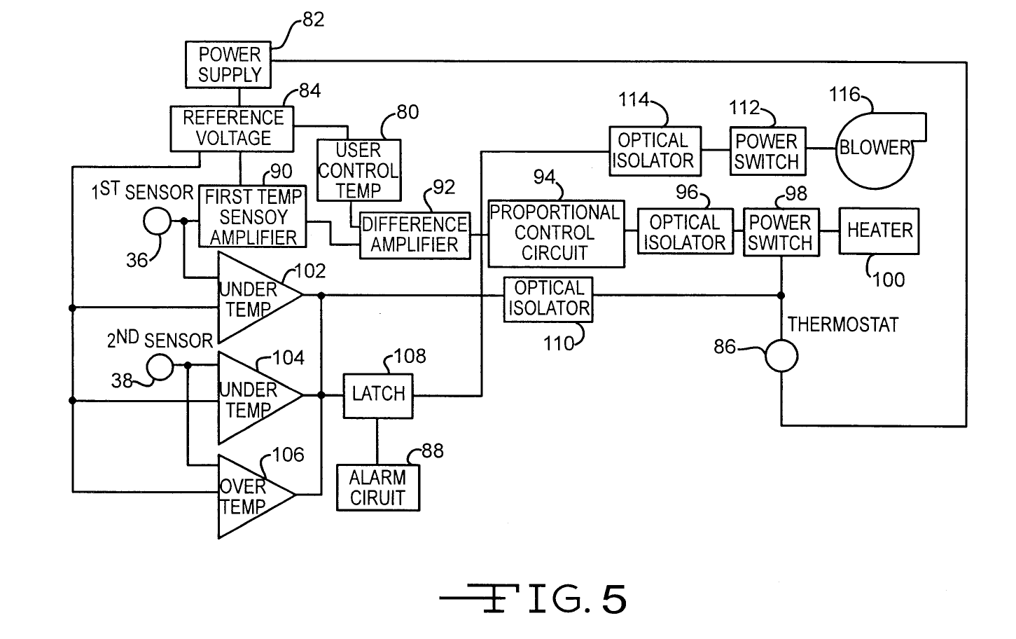

Referring to FIG. 5, one possible schematic form of a control circuit is disclosed. Other forms of temperature control circuits can be used, as can be appreciated by a person of skill in this field. The specific control circuit 34 incorporates a proportional controller that includes an alarm system to permit a servo-controlling of the warmed air to a preset temperature level that will be set by the operator or user controls 20 on the housing of the console 18. In this schematic, the user control temperature setting 80 is connected to a power supply 82 through a reference voltage circuit 84 which also provides excitation current for the first and second sensor units 36 and 38. The reference voltage circuit 84 can divide and buffer the power source 82 on the control circuit. By providing two separate sensors 36 and 38, there is a redundancy in the system, and the control circuit 34 can thereby also sense the air temperature through the second thermistor or sensor unit 38, located in proximity to the first sensor unit 36 or thermistor to thereby provide a backup for any over temperature condition. As a safety feature of this control circuit, any over temperature sensed by the second sensor 38 or under temperature sensed by either the second sensor 38 or the first sensor 36 or the opening of an over temperature thermostat 86, which can be located in the heater housing or console 18, can turn off the blanket warming system. Thus, any of these conditions of an over temperature or an under temperature will indicate a problem and can be utilized to automatically shut off the power to the heater unit 28 and the blower 26 and to also further activate audible and visual alarms in the alarm circuit 88.

The first temperature sensor 36 amplifies the sensed voltage that is proportional to the air temperature adjacent a thermal blanket that is receiving the delivered heated air. This temperature signal is amplified in a first temperature sensor amplifier 90. The amplified temperature signal is subtracted from a set point temperature from the user control temperature 80 by a differential amplifier or a difference amplifier circuit 92. The resulting output difference signal is provided to a proportional control circuit 94, and this different signal is compared to a triangular wave that is generated to provide a pulse width modulated (PWM) signal whose duty cycle is proportional to the difference in the output temperature and the set point temperature provided by the user control temperature 80. This PWM signal is then applied to a solid state power switch circuit 98 through an optical isolator 96. The power switch circuit 98 delivers appropriate pulses to the heater unit 100.

An alarm detection circuit includes under temperature comparator 102, under temperature comparator 104, and over temperature comparator 106. The output of these comparators 102, 104, and 106 are output together and inverted to be coupled to a reset input of a latch circuit 108. Additionally, the voltage across the thermostat 86 is also applied to the latch reset through an optical isolator 110. If either the first sensor thermistor 36 or the second sensor thermistor 38 senses a very low temperature, which may occur in the case of an open sensor or the second sensor 38 senses an over temperature, or if the thermostat 86 itself mechanically breaks or opens, the latch circuit 108 is reset and opens a second solid state power switch circuit 112 that is also optically isolated by an optical isolator 114. The power switch circuit 112 is in series with the heat control power switch circuit 98, and the power switch 112 controls power to the blower unit 116, as well as the heater 100, and has the capacity of shutting down the entire warming system until this alarm condition is corrected, and the warming system is reset by turning off the power and turning the power back on. The thermostat 86 is in series with both of these solid state power switches 112 and 98 and can positively interrupt power to both the heater unit 100 and the blower unit 116. The output of the latch circuit 108 can also turn on a transistor to activate both audible and visual alarms in an alarm circuit 88.

While applicants have described one embodiment of a control circuit, the embodiments of the present invention can also be operated with alternative control circuits.

### Shape Memory Alloys/Polymers

Shape memory polymers are polymers whose qualities have been altered to give them dynamic shape “memory” properties. Using thermal stimuli, shape memory polymers can exhibit a radical change from a rigid polymer to a very elastic state, and then back to a rigid state again. In its elastic state, it will recover its “memory” shape if left unrestrained. However, while pliable it can be stretched, folded or otherwise conformed to other shapes, tolerating up to 200% elongation. While manipulated, the shape memory polymer can be cooled and therefore returned to a rigid state, maintaining its manipulated shape indefinitely. This manipulation process can be repeated many times without degradation, and manufacturers can tailor most polymers with shape memory properties. An example of this polymer can be obtained from Cornerstone Research Group, Inc. of Dayton, Ohio.

A shape memory alloy is capable of remembering a previously memorized shape. It has to be deformed in its low temperature phase Martensite and subsequently heated to the high temperature phase Austenite by heated air. The alloy generates a high force during the phase transformation. The shape change is not restricted to just pure bending. A suitable actuation mode has proved to be the linear contraction of a straight wire actuator. An example of such alloy includes and is not limited to NiTi (Nickel—Titanium), CuZnAl, CuAlNi, and nitonol.

The shape memory polymer and/or alloy can be in any desired shape—including and not limited to a ribbon shape, a spiral shape, a spring shape or combinations thereof. The polymer and/or alloy can have various widths, lengths, thicknesses, treatment conditions and surfaces. The shape, size and condition depend on the desired application.

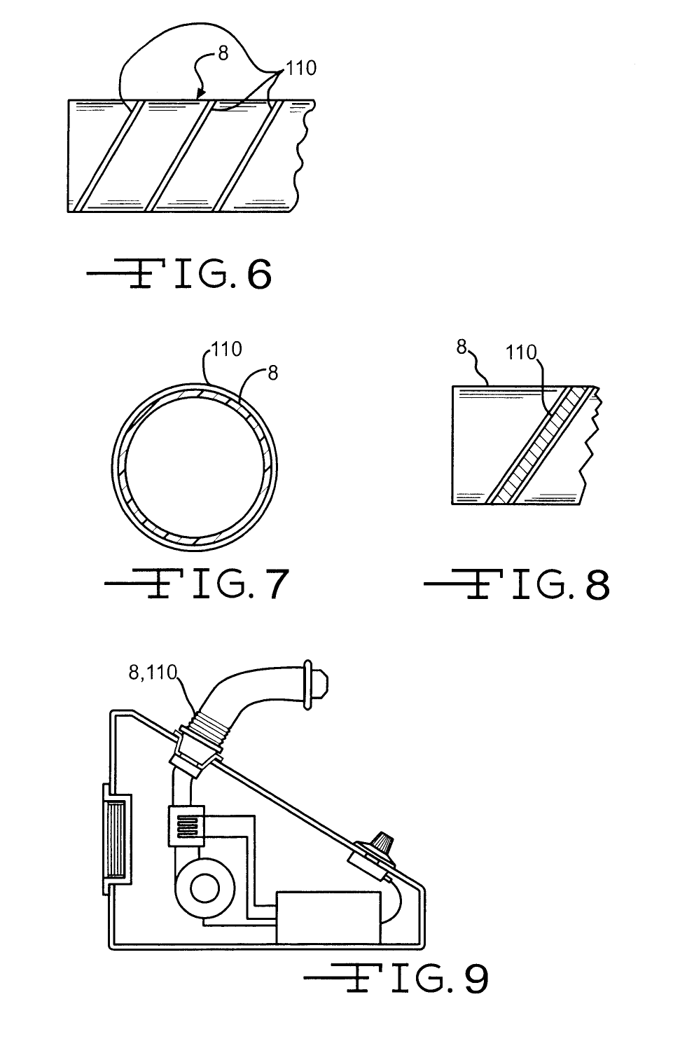

For the present invention, the shape memory polymer and/or alloy 110 attaches to the hose 8 so when the heated air passes through the hose 8 the thermal energy from the heated air contacts the shape memory polymer and/or alloy. The shape memory polymer and/or alloy 110 can be positioned on the exterior surface of the hose 8 as illustrated at FIG. 6, on the interior surface of the hose 8 as illustrated at FIG. 7, embedded in the material that forms the hose 8 as illustrated at FIG. 8, and combinations thereof. The shape memory polymer and/or alloy 110 just has to be effected by the thermal energy of the heated air passing through the conduit 8.

### A FIRST EMBODIMENT OF THE INVENTION

A point of the application is that the shape memory polymer and/or alloy 110 assists with hose 8 management by ensuring that the hose 8 is “short” and off the ground when the hose 8 is not (a) in use and (b) connected to a blanket. When the convective blower 100 is off and at normal room temperature, the shape memory polymer and/or alloy 110 with the hose 8 are in a shortened geometry as illustrated in FIG. 9. As soon as the thermal energy from the heated air is applied by switching on the blower 26 (and the heated air's thermal energy is a certain predetermined temperature), the shape memory polymer and/or alloy 110 and the hose 8 extend to its “trained” length, and may be connected to a convective warming blanket 4. Once the convective warmer 100 is switched off, the hose 8 reverts to its shortened length, and will not trail on the ground where it can pick up dirt and germs thereby constituting a potential infection control hazard.

### A SECOND EMBODIMENT OF THE INVENTION

In another embodiment, a different phase of Nitinol having a “superelastic phase” can be used to realize a spiral coiled spine. In this embodiment, the nitonol is compressed and short in the relaxed position. It may be elongated to the required length by pulling against the restoring spring force and connecting the hose 8 to the blanket 4. An external linkage mechanism could be used to maintain the hose in the extended position.

Those skilled in the art will appreciate that various adaptations and modifications of the just-described preferred embodiment can be configured without departing from the scope and spirit of the invention. Therefore, it is to be understood that, within the scope of the appended claims, the invention may be practiced other than as specifically described herein.

We claim:

1. A convective thermal unit for a patient comprising: a heater unit for heating a gas; a blower unit for forcing gas to the heater unit; a conduit for delivery of the heated gas to a receiving unit; a shape memory polymer and/or alloy material connected to the conduit so the shape memory polymer and/or alloy material is effected by the thermal energy of the heated gas;
wherein when the conduit receives the heated gas having a thermal energy at or exceeds an Austenite temperature, the conduit expands to a desired length;
wherein when the conduit receives the heated gas having a thermal energy below a Martensite temperature, the conduit compresses to a predetermined length that prevents the conduit from contacting the ground.

2. The convective thermal unit of claim 1 wherein the receiving unit is a blanket having a core for receiving a gas and a coupling port to enable the admission of gas.

3. The convective thermal unit of claim 1 further comprising a control circuit for controlling the temperature of the heater.

4. The convective thermal unit of claim 1 wherein the shape memory polymer and/or alloy is positioned on the exterior surface of the conduit, on the interior surface of the conduit, within the material that forms the conduit, and combinations thereof.

5. The convective thermal unit of claim 1 wherein the conduit expands to a desired length and desired shape when the heated gas has a thermal energy at or exceeds the Austenite temperature.

6. The convective thermal unit of claim 5 wherein the desired shape controls the turbulence in the conduit.

7. The convective thermal unit of claim 3 further comprising a sensor that measures the thermal energy of the heated gas and transmits the measurement to the control unit.

8. The convective thermal unit of claim 7 wherein the sensor is positioned in the conduit, in the convective thermal unit, in the receiving unit, or combinations thereof.

9. A convective thermal unit for a patient comprising: a heater unit for heating a gas; a blower unit for forcing gas to the heater unit; a conduit for delivery of the heated gas to a receiving unit; a shape memory polymer and/or alloy material connected to the conduit so the shape memory polymer and/or alloy material is effected by the thermal energy of the heated gas;
wherein when the conduit receives the heated gas having a thermal energy at or exceeds an Austenite temperature, the conduit compresses to a predetermined length that prevents the conduit from contacting the ground and the conduit can be extended to the receiving unit.

10. The convective thermal unit of claim 9 wherein the receiving unit is a blanket having a core for receiving a gas and a coupling port to enable the admission of gas.

11. The convective thermal unit of claim 9 further comprising a control circuit for controlling the temperature of the heater.

12. The convective thermal unit of claim 9 wherein the shape memory polymer and/or alloy is positioned on the exterior surface of the conduit, on the interior surface of the conduit, within the material that forms the conduit, and combinations thereof.

13. The convective thermal unit of claim 9 wherein the conduit expands to a desired length and desired shape when the heated gas has a thermal energy at or exceeds the Austenite temperature.

14. The convective thermal unit of claim 13 wherein the desired shape controls the turbulence in the conduit.

15. The convective thermal unit of claim 11 further comprising a sensor that measures the thermal energy of the heated gas and transmits the measurement to the control unit.

16. The convective thermal unit of claim 15 wherein the sensor is positioned in the conduit, in the convective thermal unit, in the receiving unit, or combinations thereof.
